(19) United States
(12) Patent Application Publication
(10) Pub. No.: US 2026/0023015 A1
(43) Pub. Date: Jan. 22, 2026

(54) SEMI-OPEN ATMOSPHERIC AMMONIA CONCENTRATION DETECTION SYSTEM AND METHOD FOR MEASURING ATMOSPHERIC AMMONIA CONCENTRATION

(71) Applicant: HEALTHY PHOTON (NINGBO) TECHNOLOGY CO., LTD, NINGBO CITY (CN)

(72) Inventors: Yin WANG, Ningbo City (CN); Lei RUI, Ningbo City (CN); Ting-Jung LIN, Ningbo City (CN)

(21) Appl. No.: 19/268,443

(22) Filed: Jul. 14, 2025

(30) Foreign Application Priority Data
Jul. 17, 2024 (CN) 202410955181.7

(51) Int. Cl. G01N 21/39; G01N 21/03; G01N 21/31; G01N 33/00

(52) U.S. Cl. CPC G01N 21/39; G01N 21/03; G01N 33/0006; G01N 33/0054; G01N 2021/3196

(57) ABSTRACT

The present disclosure discloses a semi-open atmospheric ammonia concentration detection system and a method for measuring an atmospheric ammonia concentration, and relates to the field of ammonia concentration measurement. By measuring and calibrating standard ammonia samples with known concentration, a reference spectrum used to reflect optical signal intensity is constructed. Direct absorption concentration corresponding to atmospheric ammonia is acquired by a data processor by using a detection signal of a photodetector in a measurement component and the reference spectrum. The detection signal is a direct absorption original spectrum corresponding to the atmospheric ammonia. High-precision detection of low-concentration ammonia is achieved.

### TECHNICAL FIELD

The present disclosure relates to the field of ammonia concentration measurement, particularly to a semi-open atmospheric ammonia concentration detection system and a method for measuring an atmospheric ammonia concentration.

### BACKGROUND

In fields such as atmospheric monitoring, environmental science, and industrial safety, there is generally a need to detect low-concentration specific components in gases, such as ammonia. Since absorption signals of low-concentration gases are weak and easily interfered by background noise and environmental factors, the concentration of the low-concentration gases cannot be accurately measured through an existing direct absorption method.

The direct absorption method is a classic spectroscopic measurement technology based on Beer-Lambert Law, which determines concentration of a gas by measuring a degree to which a gas sample absorbs light with a specific wavelength. During the specific operation, an instrument emits light with a specific wavelength that passes through a sample including a gas to be measured, and then a detector receives an optical signal after the light passes through the sample. The concentration of the gas in the sample is directly proportional to a degree of absorption of the light. However, atmospheric ammonia generally has low concentration, existing at ppb (parts per billion) or low ppm (parts per million) levels. Such low concentration means that the gas has an extremely weak absorption effect on the light, and the direct absorption method may be difficult to distinguish an effective absorption signal from the background noise, resulting in greatly reduced measurement accuracy. Atmosphere is a complex mixture, and in addition to the ammonia, also includes various components such as water vapor and dust particles, etc., all of which may interfere with absorption and scattering of the light, accordingly the absorption signal of the ammonia is further masked or confused. Therefore, most conventional direct absorption spectrometers are not designed to achieve a high sensitivity and a low noise level required to detect gases with extremely low concentration.

Secondly, environmental factors such as the temperature, pressure changes and the like may significantly affect absorption characteristics of gases, thereby affecting accuracy of measurement results. How to accurately correct measurement data under different environmental conditions to ensure comparability and consistency of the results is another issue required to be solved.

In conventional detection modes (due to strong adsorption and water solubility of the ammonia, it is difficult to use conventional gas pipeline sampling and a spectral analysis method in a closed-circuit airtight gas analysis cell), such as by use of a closed detection device, although the ammonia concentration can be measured accurately to some extent, since gas samples are required to be collected and placed in a closed environment for analysis, there are limitations such as a slow response speed and incapability to monitor the ammonia concentration in the atmospheric environment in real time.

### SUMMARY

In order to detect the ammonia concentration in the atmospheric environment in real time, the present disclosure proposes a semi-open atmospheric ammonia concentration detection system, including:
a case, the case being provided with a ventilation hole, for a gas to enter the case through the ventilation hole, and for the gas in the case to be discharged from the ventilation hole;
an ammonia concentration detector arranged in the case and configured to detect ammonia concentration in the case; the ammonia concentration detector including:
a quantum cascade laser configured to emit a laser beam and scan an atmospheric ammonia absorption line through a ramp waveform;
a reference component that forms a reference path;
a measurement component that forms a measurement path, the measurement component including a first lens, a first concave mirror, a Herriott cell, a second concave mirror, a second lens, and a photodetector;
a beam splitter configured to split the laser beam emitted by the quantum cascade laser into a first beam passing along the measurement path and a second beam passing along the reference path; the first beam passing through the first lens, the first concave mirror, the Herriott cell, the second concave mirror, and the second lens in sequence; and a data processor;
the measurement component being configured to detect and process a signal of an ammonia characteristic absorption line that passes through the second lens;
the reference component being configured to detect and process a spectral signal of the second beam passing through the reference path;
the data processor being configured to acquire a direct absorption concentration of a detected gas based on the signals processed by the measurement component and the reference component;
a fan arranged in the case, the fan being capable of accelerating a flow speed of the gas entering and exiting the case when turned on; and
a power supply module configured in the case, electrically connected to the ammonia concentration detector and the fan, and capable of supplying power to the ammonia concentration detector and the fan.

Further, an inner surface of the case is provided with a lining layer that prevents ammonia gas adsorption.

The present disclosure further proposes a method for measuring an atmospheric ammonia concentration, applied to the atmospheric ammonia concentration detection system as described above. The measuring method includes:
emitting the laser beam through the quantum cascade laser and scanning an ammonia absorption line in a standard ammonia sample through a ramp waveform;
detecting, through the photodetector in the measurement component, the standard ammonia sample that passes through the second lens, capturing a characteristic absorption line signal of the standard ammonia sample, and processing the signal to obtain a direct absorption original spectrum;
demodulating, through the data processor, the direct absorption original spectrum by using a wavelength modulation spectroscopy (WMS) modulation and demodulation algorithm to obtain a WMS modulation and demodulation absorption spectrum, performing quadratic fitting on the WMS modulation and demodulation absorption spectrum to obtain a baseline spectrum, and acquiring spectral data after the baseline spectrum is subtracted from the direct absorption original spectrum;
acquiring, through the data processor, a direct absorption concentration of a standard ammonia in a current temperature and pressure environment by using the direct absorption spectrum and an absorption spectrum with preset concentration in a standard temperature and pressure environment, and performing, through a WMS wavelength modulation and demodulation spectrum, range calibration on the direct absorption concentration in the current temperature and pressure environment, to obtain a reference spectrum that reflects optical signal intensity; and
acquiring, through the data processor, direct absorption concentration corresponding to atmospheric ammonia by using a detection signal of the photodetector in the measurement component and the reference spectrum, the detection signal being a direct absorption original spectrum corresponding to the atmospheric ammonia.

Further, the photodetector in the measurement component includes: an analog-to-digital converter and a field-programmable gate array (FPGA)-based lock-in amplifier.

The detecting, through the photodetector in the measurement component, the standard ammonia sample that passes through the second lens, capturing the characteristic absorption line signal of the standard ammonia sample, and processing the signal to obtain the direct absorption original spectrum includes:
detecting, through the photodetector in the measurement component, the standard ammonia sample that passes through the second lens, capturing the characteristic absorption line signal of the standard ammonia sample to obtain an absorption spectrum, enhancing a signal of the absorption spectrum through the lock-in amplifier, and sampling, by using the analog-to-digital converter, an enhanced absorption spectrum to obtain the direct absorption original spectrum.

Further, the performing quadratic fitting on the WMS modulation and demodulation absorption spectrum to obtain the baseline spectrum includes:
identifying a peak position of the WMS modulation and demodulation absorption spectrum, and defining a left region and a right region of the WMS modulation and demodulation absorption spectrum through the peak position;
performing quadratic fitting on the spectral data in the left region and the right region respectively by using a quadratic polynomial fitting method, to obtain a first fitting curve corresponding to the left region and a second fitting curve corresponding to the right region; and
smoothly connecting the first fitting curve and the second fitting curve to obtain the baseline spectrum that is not affected by standard ammonia absorption.

Further, the acquiring the direct absorption concentration of the standard ammonia in the current temperature and pressure environment includes:
obtaining a K value by linearly fitting the direct absorption spectrum and the absorption spectrum with the preset concentration in the standard temperature and pressure environment;
acquiring a direct absorption concentration of the standard ammonia in the standard temperature and pressure environment through the K value;

acquiring a temperature and pressure compensation coefficient table through a simulation program;
searching the temperature and pressure compensation coefficient table for a corresponding compensation coefficient M according to a temperature and an air pressure in the current temperature and pressure environment; and
calculating the direct absorption concentration of the standard ammonia in the current temperature and pressure environment through the compensation coefficient M and the direct absorption concentration of the standard ammonia in the standard temperature and pressure environment.

Further, the acquiring, through the data processor, direct absorption concentration corresponding to atmospheric ammonia by using the detection signal of the photodetector in the measurement component and the reference spectrum includes:
emitting a laser beam through the quantum cascade laser and scanning the atmospheric ammonia absorption line through the ramp waveform, the ramp waveform including a plurality of scanning points;
detecting, through the photodetector in the measurement component, the atmospheric ammonia that passes through the second lens, capturing a characteristic absorption line signal of the atmospheric ammonia, and processing the signal to obtain the direct absorption original spectrum;
demodulating, through the data processor, the direct absorption original spectrum processed by the measurement component by using the WMS modulation and demodulation algorithm to obtain a demodulation signal corresponding to each scanning point, and normalizing the demodulation signal corresponding to each scanning point to obtain a normalized spectrum, the demodulation signals including: a sinusoidal signal 1f and a second harmonic signal 2f; and
projecting the normalized spectrum to the reference spectrum through the data processor, and acquiring a direct absorption concentration corresponding to the current normalized spectrum through a data ratio.

Further, the projecting the normalized spectrum to the reference spectrum through the data processor, and obtaining the direct absorption concentration corresponding to the current normalized spectrum through the data ratio includes:
acquiring absorbance of a maximum absorption peak in the reference spectrum;
acquiring a characteristic absorption peak in the normalized spectrum, and acquiring absorbance corresponding to the characteristic absorption peak;
establishing a proportional relation based on the absorbance of the maximum absorption peak in the reference spectrum, the direct absorption concentration corresponding to the reference spectrum, and the absorbance corresponding to the characteristic absorption peak in the normalized spectrum, an expression of the proportional relation being

$$\frac{{S}_{A}}{{S}_{a c}} = \frac{{C}_{A}}{{C}_{a c}} ,$$

where S denotes the reference spectrum, S_{A} denotes the absorbance of the maximum absorption peak in the reference spectrum, S_{ac} denotes the direct absorption concentration corresponding to the reference spectrum, C denotes the normalized spectrum, C_{A} denotes the absorbance corresponding to the characteristic absorption peak in the normalized spectrum, and C_{ac} denotes direct absorption concentration corresponding to a to-be-solved normalized spectrum; and
solving the direct absorption concentration corresponding to the to-be-solved normalized spectrum through the proportional relation.

Further, the reference component includes: a high-concentration ammonia reaction cell and a photodetector that includes an analog-to-digital converter and a lock-in amplifier. The photodetector in the reference component is configured to detect the second beam passing through the high-concentration ammonia reaction cell to obtain an absorption spectrum corresponding to the reference component, enhance a signal of the absorption spectrum through the lock-in amplifier, and sample, by using the analog-to-digital converter, an enhanced absorption spectrum to obtain a direct absorption original spectrum.

The measuring method further includes:
locking, by the data processor, an optimal current value by using the direct absorption original spectrum processed by the measurement component and the reference component respectively, and controlling a drive current of the quantum cascade laser with the optimal current value.

Further, the locking, by the data processor, the optimal current value by using the direct absorption original spectrum processed by the measurement component and the reference component respectively, and controlling the drive current of the quantum cascade laser with the optimal current value includes:
regulating an operating drive current of the quantum cascade laser, to allow the operating drive current to increase from a preset minimum current until the operating drive current reaches a preset safe limit current;
acquiring the direct absorption original spectrum detected by the measurement component during the regulation as a first absorption spectrum, and the direct absorption original spectrum detected by the reference component as a second absorption spectrum; acquiring maximum absorption peaks of the first absorption spectrum and the second absorption spectrum;
calculating target absorbance by using an optical signal intensity of the maximum absorption peak in the first absorption spectrum and an optical signal intensity of the maximum absorption peak in the second absorption spectrum; a calculation formula for the target absorbance being as follows:

$$A = - {log}_{10} ( \frac{{I}_{m e a s u r e}}{{I}_{reference}} ) ,$$

where Iₘₑₐₛᵤᵣₑ denotes the optical signal intensity of the maximum absorption peak in the first absorption spectrum, I_{reference} denotes the optical signal intensity of the maximum absorption peak in the second absorption spectrum, and A denotes the target absorbance; and
locking a current corresponding to the target absorbance as the optimal current value, and controlling the drive current of the quantum cascade laser with the optimal current value.

Compared with the existing technologies, the present disclosure achieves at least the following beneficial effects:

(1) Through the semi-open atmospheric ammonia concentration detection system in the present disclosure, ammonia concentration can be directly detected in situ (that is, in an atmospheric environment) in real time, a cumbersome process of conventional sampling-transportation-laboratory analysis is eliminated. According to the present disclosure, changes in ammonia concentration in a monitoring region can be instantly fed back, and dynamic changes in the atmospheric ammonia can be continuously measured, which is adapted to the long-term environmental monitoring and trend analysis, prevents contamination or concentration changes that may be introduced during sampling, transportation, and storage, and improves accuracy and reliability of measurement results.

(2) According to the present disclosure, the direct absorption original spectrum processed by the measurement component is demodulated by using the WMS modulation and demodulation algorithm, which increases a signal-to-noise ratio of the signal. At the same time, a signal-to-noise ratio of actual measurement data is enhanced through a data ratio, thereby achieving high-precision detection of low-concentration ammonia.

(3) According to the present disclosure, the absorbance of the maximum absorption peak in the reference spectrum is acquired; the characteristic absorption peak in the normalized spectrum is acquired, and the absorbance corresponding to the characteristic absorption peak is acquired; the proportional relation is established based on the absorbance of the maximum absorption peak in the reference spectrum, the direct absorption concentration corresponding to the reference spectrum, and the absorbance corresponding to the characteristic absorption peak in the normalized spectrum; and the direct absorption concentration corresponding to the to-be-solved normalized spectrum is solved through the proportional relation. That is, according to the present disclosure, by comparing the real-time measured normalized spectrum to the reference spectrum, a proportional relation can be determined. The proportional relation reflects a proportional relationship between an ammonia concentration under an actual measurement condition and an ammonia concentration under a reference condition. Therefore, even if a direct absorption signal is very weak, the actual concentration of the atmospheric ammonia can still be accurately analyzed from weak signal changes through this ratio analysis method, thereby improving measurement accuracy. The method essentially uses the known and highly calibrated benchmark data (the reference spectrum) as a basis, and the signal-to-noise ratio of the actual measurement data is enhanced through the mathematical comparison, thereby implementing high-precision detection of low-concentration ammonia.

(4) According to the present disclosure, when the standard ammonia sample with known concentration is measured and calibrated, the K value is obtained by linearly fitting the direct absorption spectrum and the absorption spectrum with the preset concentration in the standard temperature and pressure environment; the direct absorption concentration of the standard ammonia in the standard temperature and pressure environment is acquired through the K value; the temperature and pressure compensation coefficient table is searched for the corresponding compensation coefficient M according to the temperature and the air pressure in the current temperature and pressure environment; and the direct absorption concentration of the standard ammonia in the current temperature and pressure environment is calculated through the compensation coefficient M and the direct absorption concentration of the standard ammonia in the standard temperature and pressure environment. That is, according to the present disclosure, the influence of environmental factors on the measurement results is corrected through the calculated K value, thereby accurately assessing the true concentration (direct absorption concentration) of the standard ammonia in the standard temperature and pressure environment, and the direct absorption concentration of the standard ammonia under the current temperature and pressure environment is calculated through the compensation coefficient M and the true concentration, thereby eliminating the influence of environmental variables on the calculation of the direct absorption concentration and improving accuracy of the calibration.

(5) According to the present disclosure, quadratic fitting is performed on the WMS modulation and demodulation absorption spectrum to obtain the baseline spectrum, the spectral data after the baseline spectrum is subtracted from the direct absorption original spectrum is acquired, and the direct absorption spectrum is obtained. That is, according to the present disclosure, by subtracting the baseline spectrum from the direct absorption original spectrum (separating a pure ammonia gas absorption signal), an influence of background changes is removed from original data, so that the remaining signals mainly reflect absorption characteristics of ammonia, improving accuracy and sensitivity of the measurement.

(6) Performance of the quantum cascade laser includes the optical output power and the stability, which varies with the operating drive current. Therefore, in the present disclosure, the optimal current value is locked through the data processor by using the direct absorption original spectrum processed by the measurement component and the reference component respectively, and the drive current of the quantum cascade laser is controlled with the optimal current value, so that the quantum cascade laser operates in an optimal state to obtain a strongest and stable absorption signal, thereby improving efficiency and accuracy of the measurement.

(7) According to the present disclosure, by comparison with a reference spectrum with a high signal-to-noise ratio and analysis with a data ratio, signal changes can be effectively amplified, thereby implementing accurate measurement of the atmospheric ammonia concentration under low concentration conditions.

(8) According to the present disclosure, by normalizing the demodulation signal corresponding to each scanning point, signal noise and nonlinear effects are eliminated, and the accuracy of the measurement is improved.

### BRIEF DESCRIPTION OF THE DRAWINGS

FIG. 1 is a schematic structural diagram of an ammonia concentration detector according to an embodiment.

FIG. 2 is a schematic diagram of a direct absorption original spectrum according to an embodiment.

FIG. 3 is a schematic diagram of a WMS modulation and demodulation absorption spectrum according to an embodiment.

FIG. 4 is a schematic diagram of a direct absorption spectrum according to an embodiment.

FIG. 5 is a schematic diagram of a three-dimensional array corresponding to a temperature and pressure compensation coefficient table according to an embodiment.

FIG. 6 is a schematic structural diagram of a semi-open atmospheric ammonia concentration detection system according to an embodiment.

### REFERENCE SIGNS

1, quantum cascade laser; 2, beam splitter; 3, first lens; 4, first concave mirror; 5, Herriott cell; 6, second concave mirror; 7, second lens; 8, photodetector in measurement component; 9, high-concentration ammonia reaction cell; 10, photodetector in reference component; 11, data processor; 12, case; 13, ammonia concentration detector; 14, fan; 15, solar panel; 16, power control module; 17, lining layer; 18, ventilation hole.

### DETAILED DESCRIPTION OF THE EMBODIMENTS

The technical solution of the present disclosure is further described below with reference to specific embodiments of the present disclosure combined with the accompanying drawings, but the present disclosure is not limited to these embodiments.

### Embodiment I

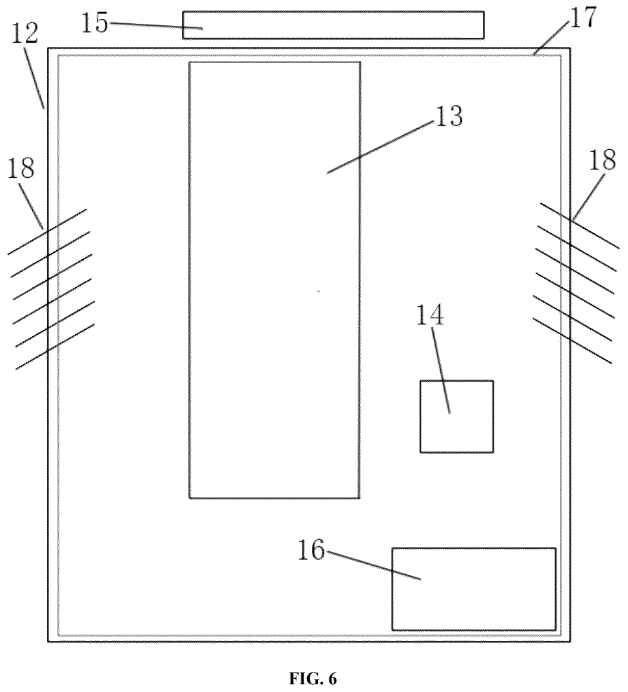

In order to ensure normal operation of an ammonia concentration detector in harsh outdoor environments, in the embodiment of the present disclosure, a semi-open atmospheric ammonia concentration detection system is provided, as shown in FIG. 6, which may include a case 12 and an ammonia concentration detector 13.

The case 12 is provided with a ventilation hole 18, a gas is capable of entering the case 12 through the ventilation hole 18, and the gas in the case 12 is capable of being discharged from the ventilation hole 18. In addition, the front of the case 12 is also designed with an openable case door to facilitate placement or removal of an object in the case 12.

The ammonia concentration detector 13 is arranged in the case 12 and configured to detect ammonia concentration in the case 12. Specifically, the ammonia concentration detector 13 may be vertically or horizontally mounted inside the case 12.

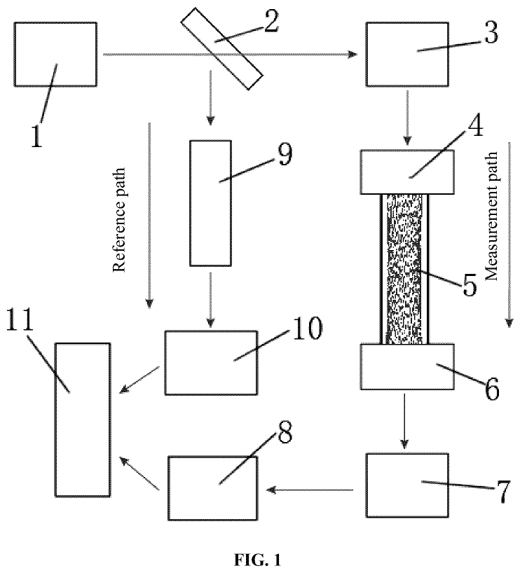

As shown in FIG. 1, the ammonia concentration detector 13 may include:
a quantum cascade laser 1 configured to emit a laser beam and scan an atmospheric ammonia absorption line through a ramp waveform;
a reference component that forms a reference path;
a measurement component that forms a measurement path, in which the measurement component includes a first lens 3, a first concave mirror 4, a Herriott cell 5, a second concave mirror 6, a second lens 7, and a photodetector 8;
a beam splitter 2 configured to split the laser beam emitted by the quantum cascade laser 1 into a first beam passing along the measurement path and a second beam passing along the reference path; the first beam passes through the first lens 3, the first concave mirror 4, the Herriott cell 5, the second concave mirror 6, and the second lens 7 in sequence; and
a data processor 11;
the measurement component is configured to detect and process a signal of an ammonia characteristic absorption line that passes through the second lens 7;
the reference component is configured to detect and process a spectral signal of the second beam passing through the reference path;
the data processor 11 is configured to obtain direct absorption concentration of a detected gas based on the signals processed by the measurement component and the reference component.

The system may further include a fan 14 arranged in the case 12, the fan 14 is capable of accelerating a flow speed of the gas entering and exiting the case 12 when turned on. Specifically, a mounting region of the fan 14 is provided with the ventilation hole or a ventilation slot, and the fan 14 accelerates a circulation of air inside and outside the case 12.

The system may further include a power supply module which is provided in the case 12 and electrically connected to the ammonia concentration detector 13 and the fan 14, and capable of supplying power to the ammonia concentration detector 13 and the fan 14.

Specifically, the power supply module includes: a power control module 16 and a solar panel 15 mounted on a top portion of the case 12. The solar panel 15 is configured to convert solar energy into electrical energy and store the electrical energy in the power control module 16. The power control module 16 is configured to provide power for the ammonia concentration detector 13 and the fan 14. At the same time, the power control module 16 is equipped with a battery pack with an optional capacity.

In the embodiment, a battery life of the atmospheric ammonia concentration detection system is improved through the solar panel 15.

In particular, considering easy adsorption and corrosiveness of the ammonia, a lining layer 17 that prevents ammonia adsorption is designed on an inner surface of the case 12. The lining layer 17 is made of Teflon (or other materials that are not easily adsorbed by the ammonia), which effectively reduces occurrence of inaccurate detection data due to excessive ammonia adsorbed by the inner wall.

In addition, it should be noted that when a reference spectrum is required to be acquired (that is, when range calibration is performed on the standard ammonia), the fan 14 is required to be turned off. When an actual measurement is required (that is, when the atmospheric ammonia is required to be measured), the measurement can only be started by turning on the fan 14 to make an atmospheric environment inside the case 12 consistent with an external environment, and the fan is required to be remain on during the measurement.

With the semi-open atmospheric ammonia concentration detection system in the present disclosure, the ammonia concentration can be directly detected in situ (that is, in an atmospheric environment) in real time, a cumbersome process of conventional sampling-transportation-laboratory analysis is eliminated. In the embodiment, changes in the ammonia concentration in a monitoring region can be instantly fed back, and dynamic changes in the atmospheric ammonia can be continuously measured, which is adapted to long-term environmental monitoring and trend analysis, prevents contamination or concentration changes that may be introduced during the sampling, transportation, and storage, and improves accuracy and reliability of measurement results.

In addition, in the atmospheric ammonia concentration detection system provided in the present disclosure, the ammonia concentration detector 13 is mounted in the case 12, which reduces contamination of a mirror surface by rainwater, gravel, and dust, and prevents an influence of environmental factors on accuracy of the detection data. At the same time, in the atmospheric ammonia concentration detection system provided in the embodiment, high-power devices such as an air pump, a water pump, etc., are not used, and power of the entire machine is lower than that of similar products on the market.

### Embodiment II

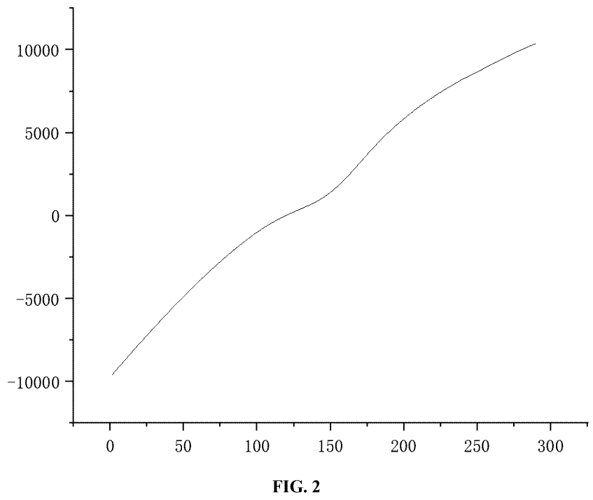

Atmospheric ammonia has very low natural concentration, and it is very difficult to extract ammonia concentration information directly from an original absorption spectrum because a signal strength is extremely weak and may be drowned in noise. In order to address the technical problem, the present disclosure provides a method for measuring atmospheric ammonia concentration, applied to the semi-open atmospheric ammonia concentration detection system as shown in FIG. 6. The measuring method may include:
a laser beam is emitted through the quantum cascade laser 1, and an ammonia absorption line in a standard ammonia sample is scanned through a ramp waveform;
the laser beam is split, by the beam splitter 2, into a first beam passing along the measurement path and a second beam passing along the reference path; the first beam passes through the first lens 3, the first concave mirror 4, the Herriott cell 5, the second concave mirror 6, and the second lens 7 in sequence; and
the standard ammonia sample passing through the second lens 7 is detected by the photodetector 8 in the measurement component, a characteristic absorption line signal of the standard ammonia sample is captured, and the characteristic absorption line signal is processed to obtain the direct absorption original spectrum as shown in FIG. 2 (ordinate of the spectrum represents absorption concentration data, and abscissa of the spectrum represents time sequence numbers).

The photodetector in the measurement component includes: an analog-to-digital converter and a FPGA-based lock-in amplifier.

The step of detecting, by the photodetector 8 in the measurement component, the standard ammonia sample passing through the second lens 7, capturing the characteristic absorption line signal of the standard ammonia sample, and processing the characteristic absorption line signal to obtain the direct absorption original spectrum may specifically include:
the standard ammonia sample passing through the second lens 7 is detected by the photodetector 8 in the measurement component, the characteristic absorption line signal of the standard ammonia sample is captured to obtain an absorption spectrum, a signal of the absorption spectrum is enhanced by the lock-in amplifier, and an enhanced absorption spectrum is sampled by using the analog-to-digital converter to obtain the direct absorption original spectrum.

It is to be explained that the absorption spectrum refers to a spectrum formed by changes in light intensity due to absorption of light with a specific wavelength by ammonia molecules after the first beam passes through the standard ammonia sample. The beam emitted by the quantum cascade laser passes through the open Herriott cell and is reflected multiple times therein. In this process, the ammonia molecules may absorb the light of the specific wavelength, causing changes in intensity of the transmitted light, and the transmitted light is eventually captured by the photodetector 8 in the measurement component.

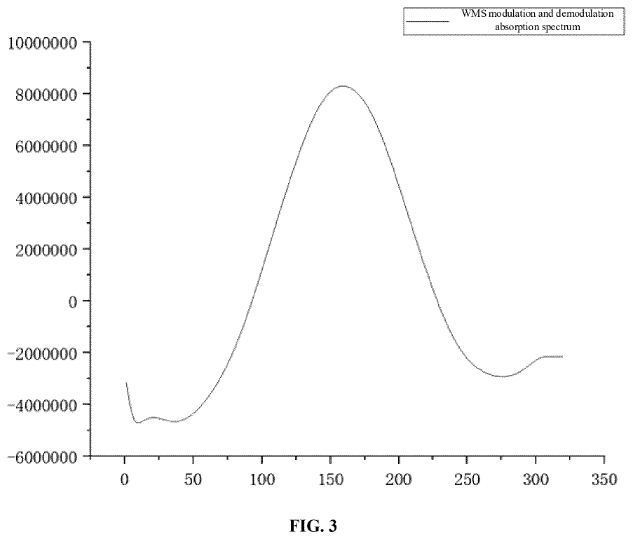

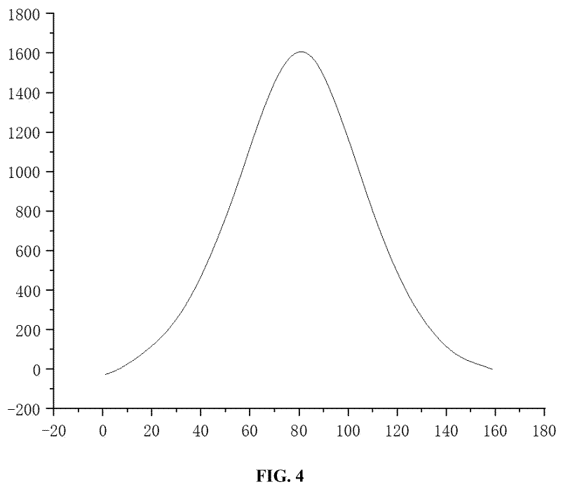

The direct absorption original spectrum is demodulated by the data processor 11 by using a wavelength modulation spectroscopy (WMS) modulation and demodulation algorithm, to obtain a WMS modulation and demodulation absorption spectrum as shown in FIG. 3 (the ordinate of the spectrum represents normalized data of a sinusoidal signal 1f and a second harmonic signal 2f, and the abscissa of the spectrum represents time sequence numbers), quadratic fitting is performed on the WMS modulation and demodulation absorption spectrum to obtain a baseline spectrum, and spectral data after the baseline spectrum is subtracted from the direct absorption original spectrum is acquired, and the direct absorption spectrum is obtained, as shown in FIG. 4 (the ordinate of the spectrum represents direct absorption concentration data, and the abscissa of the spectrum represents time sequence numbers).

The step of performing the quadratic fitting on the WMS modulation and demodulation absorption spectrum to obtain the baseline spectrum may specifically include:
a peak position of the WMS modulation and demodulation absorption spectrum is identified, and a left region and a right region of the WMS modulation and demodulation absorption spectrum are defined through the peak position;
the quadratic fitting is performed on spectral data in the left region and the right region respectively by a quadratic polynomial fitting method, to obtain a first fitting curve corresponding to the left region and a second fitting curve corresponding to the right region; and
the first fitting curve is smoothly connected to the second fitting curve to obtain the baseline spectrum that is not affected by standard ammonia absorption.

It should be explained that the direct absorption original spectrum includes an absorption signal of a target gas (ammonia) and non-absorption related background signals (such as environmental scattering and instrument noise, etc.). An absorption characteristic of the target gas is generally located in a specific region of the spectrum. In order to focus only on this region and reduce influences of other factors, left and right boundaries of the region are first required to be determined, that is, it is determined which data points belong to the left region or the right region of the spectrum, and the quadratic polynomial fitting is performed on the spectral data of the left region and the right region. The quadratic polynomial fitting is a curve-fitting technology that describes a general trend of these data points by minimizing a sum of squared errors to find an optimal fitting curve. In the present disclosure, a purpose is to construct a smooth curve that represents a background signal or a baseline that is not affected by the absorption of the target gas. This baseline reflects non-absorptive changes in the spectrum, such as light source fluctuations. The operation of subtracting the baseline spectrum obtained through the quadratic fitting from the direct absorption original spectrum is essentially to remove the effect of background changes from the original data, making the remaining signals mainly reflect the absorption characteristic of the target gas, improving the accuracy and sensitivity of the analysis.

Direct absorption concentration of the standard ammonia in a current temperature and pressure environment is acquired through the data processor 11 by using the direct absorption spectrum and an absorption spectrum with preset concentration in a standard temperature and pressure environment, and a range calibration is performed on the direct absorption concentration in the current temperature and pressure environment through the WMS wavelength modulation and demodulation spectrum, to obtain a reference spectrum that reflects an optical signal intensity (2f/1f).

It should be noted that in the embodiment, an absorption spectrum with a preset concentration in the standard temperature and pressure environment is pre-stored data, and pre-stored data thereof includes: a standard temperature temp_α, a standard air pressure pressure_α, and a preset concentration value of the absorption spectrum 2 ppm.

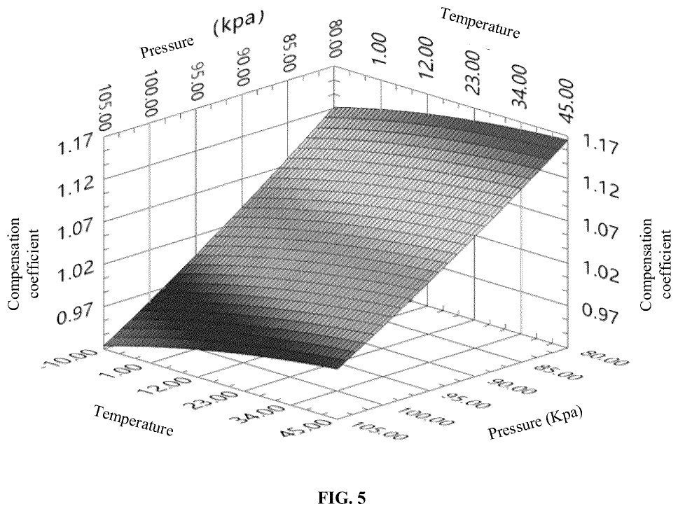

The step of acquiring the direct absorption concentration of the standard ammonia in the current temperature and pressure environment specifically includes:
a K value is obtained by linearly fitting the direct absorption spectrum and the absorption spectrum with the preset concentration in the standard temperature and pressure environment;
the direct absorption concentration of the standard ammonia in the standard temperature and pressure environment is acquired through the K value, an acquisition formula is:
   the direct absorption concentration=2 ppm/K value;
a temperature and pressure compensation coefficient table is acquired through a labview simulation program; it should be noted that the temperature and pressure compensation coefficient table is stored in a three-dimensional array, and the three-dimensional array is shown in FIG. 5;
a corresponding compensation coefficient M is searched for in the temperature and pressure compensation coefficient table according to a temperature and an air pressure in the current temperature and pressure environment;
the direct absorption concentration of the standard ammonia in the current temperature and pressure environment is calculated according to the compensation coefficient M and the direct absorption concentration of the standard ammonia in the standard temperature and pressure environment; a calculation formula is as follows:
   direct absorption concentration in current temperature and pressure environment=direct absorption concentration in standard temperature and pressure environment×M=2 ppm/K value×M; and
the direct absorption concentration corresponding to atmospheric ammonia is acquired through the data processor 11 by using a detection signal of the photodetector 8 in the measurement component and the reference spectrum; the detection signal is a direct absorption original spectrum corresponding to the atmospheric ammonia.

The step of acquiring, through the data processor 11 by using the detection signal of the photodetector 8 in the measurement component and the reference spectrum, the direct absorption concentration corresponding to atmospheric ammonia specifically includes:
a laser beam is emitted through the quantum cascade laser 1, and the atmospheric ammonia absorption line is scanned through the ramp waveform; the ramp waveform includes a plurality of scanning points;
the laser beam is split by the beam splitter 2 into a first beam passing along the measurement path and a second beam passing along the reference path; and
the atmospheric ammonia passing through the second lens 7 is detected by the photodetector 8 in the measurement component, a characteristic absorption line signal of the atmospheric ammonia is captured, and the characteristic absorption line signal is processed to obtain the direct absorption original spectrum, which specifically includes:
a signal of the ammonia absorption line passing through the second lens is detected by the photodetector 8 in the measurement component to obtain the absorption spectrum, a signal of the absorption spectrum is enhanced by the lock-in amplifier, and an enhanced absorption spectrum is sampled by the analog-to-digital converter to obtain the direct absorption original spectrum.

It should be noted that, in the embodiment, after a drive current of the quantum cascade laser 1 is controlled with an optimal current value (that is, after the ammonia concentration detector operates stably), the signal of the ammonia absorption line passing through the second lens 7 is processed.

The direct absorption original spectrum obtained by processing by the measurement component is demodulated through the data processor 11 by using the direct absorption original spectrum to obtain a demodulation signal corresponding to each scanning point, and the demodulation signal corresponding to each scanning point is normalized to obtain a normalized spectrum. The demodulation signal includes: a sinusoidal signal 1f and a second harmonic signal 2f.

The normalized spectrum is projected to the reference spectrum through the data processor 11, and the direct absorption concentration corresponding to the current normalized spectrum is acquired through a data ratio, which specifically includes:
absorbance of a maximum absorption peak in the reference spectrum is acquired;
a characteristic absorption peak in the normalized spectrum is acquired, and absorbance corresponding to the characteristic absorption peak is acquired;
a proportional relation is established based on the absorbance of the maximum absorption peak in the reference spectrum, the direct absorption concentration corresponding to the reference spectrum, and the absorbance corresponding to the characteristic absorption peak in the normalized spectrum; an expression of the proportional relation is

$$\frac{{S}_{A}}{{S}_{a c}} = \frac{{C}_{A}}{{C}_{a c}} ,$$

where S denotes the reference spectrum, S_{A} denotes the absorbance of the maximum absorption peak in the reference spectrum, S_{ac} denotes the direct absorption concentration corresponding to the reference spectrum, C denotes the normalized spectrum, C_{A} denotes the absorbance corresponding to the characteristic absorption peak (maximum absorption peak) in the normalized spectrum, and C_{ac} denotes the direct absorption concentration corresponding to a to-be-solved normalized spectrum; and
the direct absorption concentration corresponding to the to-be-solved normalized spectrum is solved through the proportional relation.

The atmospheric ammonia has very low natural concentration, and it is very difficult to extract the ammonia concentration information directly from the original absorption spectrum because the signal strength is extremely weak and may be drowned in noise. In this regard, in the present disclosure, the acquired normalized spectrum (2f/1f) is compared to or projected to the reference spectrum, and the atmospheric ammonia concentration is solved through the proportional relationship between the acquired normalized spectrum and the reference spectrum, which solves the problem of difficulty in accurately measuring low-concentration ammonia.

It is to be explained that, according to the present disclosure, by comparing the real-time measured normalized spectrum to the reference spectrum, a proportional relation can be determined. The proportional relation reflects a proportional relationship between an ammonia concentration under an actual measurement condition and an ammonia concentration under a reference condition. Therefore, even if a direct absorption signal is very weak, the actual concentration of the atmospheric ammonia can still be accurately analyzed from weak signal changes through the ratio analysis method, thereby improving the measurement accuracy. The method essentially uses known and highly calibrated benchmark data (the reference spectrum) as a basis, and the signal-to-noise ratio of the actual measurement data is enhanced through a mathematical comparison, thereby achieving high-precision detection of low-concentration ammonia.

In the embodiment, the reference component includes: a high-concentration ammonia reaction cell 9 and a photodetector 10 that includes an analog-to-digital converter and a lock-in amplifier. The photodetector 10 in the reference component is configured to detect the second beam passing through the high-concentration ammonia reaction cell 9 to obtain an absorption spectrum corresponding to the reference component, enhance a signal of the absorption spectrum through the lock-in amplifier, and sample, by using the analog-to-digital converter, an enhanced absorption spectrum to obtain the direct absorption original spectrum.

The measuring method may further include:
an optimal current value is locked through the data processor 11 by using the direct absorption original spectrum processed by the measurement component and the reference component respectively, and the drive current of the quantum cascade laser 1 is controlled with the optimal current value, which specifically includes:
an operating drive current of the quantum cascade laser 1 is regulated so that the operating drive current increases from a preset minimum current until the operating drive current reaches a preset safe limit current;
the direct absorption original spectrum detected by the measurement component during the regulation is acquired as a first absorption spectrum, and the direct absorption original spectrum detected by the reference component is acquired as a second absorption spectrum;
maximum absorption peaks of the first absorption spectrum and the second absorption spectrum are acquired;
a target absorbance is calculated through an optical signal intensity of the maximum absorption peak in the first absorption spectrum and an optical signal intensity of the maximum absorption peak in the second absorption spectrum; a calculation formula for the target absorbance is as follows:

$$A = - {log}_{10} ( \frac{{I}_{m e a s u r e}}{{I}_{reference}} ) ,$$

where Iₘₑₐₛᵤᵣₑ denotes the optical signal intensity of the maximum absorption peak in the first absorption spectrum, I_{reference} denotes the optical signal intensity of the maximum absorption peak in the second absorption spectrum, and A denotes the target absorbance; and
a current corresponding to the target absorbance is locked as the optimal current value, and the drive current of the quantum cascade laser is controlled with the optimal current value.

The performance of the quantum cascade laser includes an optical output power and a stability, which varies with the operating drive current. Therefore, in the present disclosure, the optimal current value is locked through the data processor by using the direct absorption original spectrum processed by the measurement component and the reference component respectively, and the drive current of the quantum cascade laser is controlled with the optimal current value, so that the quantum cascade laser operates in an optimal state to obtain the strongest and stable absorption signal, thereby improving efficiency and accuracy of the measurement.

It is to be noted that all directional indications (such as up, down, left, right, front, and back) in implementations of the present disclosure are only used to explain a relative position relationship, motion state, or the like of each component in a certain specific posture (as illustrated in the figures). If the specific posture changes, a directional indication changes accordingly. In addition, descriptions such as “first”, “second”, “one”, and the like in the present disclosure are for descriptive purposes only and cannot be understood as indicating or implying relative importance thereof or implicitly indicating the number of indicated technical features. Therefore, features defined with “first” and “second” may explicitly or implicitly include at least one of these features. In the description of the present disclosure, “a plurality of” means two or more, such as two or three, unless expressly and specifically stated otherwise. In the present disclosure, unless expressly stated and limited, the terms “connect” and “fix” should be understood in a broad sense, such as, a fixed connection, a detachable connection, or an integral connection; a mechanical connection, or an electrical connection; a direct connection, an indirect connection through an intermediate medium, an internal connection of two elements, or an interaction of two elements. For those of ordinary skill in the art, the specific meanings of the foregoing terms in the present disclosure can be understood according to specific circumstances. In addition, the technical solutions of various embodiments of the present disclosure can be combined with each other, which must be based on what those of ordinary skill in the art can realize. When a combination of the technical solutions is contradictory or cannot be realized, it should be considered that such a combination of the technical solutions does not exist and is not within the protection scope required by the present disclosure.

1. A semi-open atmospheric ammonia concentration detection system, comprising:
a case with at least one ventilation hole;
an ammonia concentration detector arranged in the case and configured to detect ammonia concentration, the ammonia concentration detector comprising:
a quantum cascade laser configured to emit a laser beam and scan an atmospheric ammonia absorption line;
a reference component that forms a reference path, the reference component comprising a high-concentration ammonia reaction cell and a reference photodetector;
a measurement component that forms a measurement path, the measurement component comprising a first mirror, a first concave mirror, a Herriott cell, a second concave mirror, a second mirror, and a measurement photodetector;
a beam splitter configured to split the laser beam emitted by the quantum cascade laser into a first beam passing along the measurement path and a second beam passing along the reference path, the first beam passing through the first mirror, the first concave mirror, the Herriott cell, the second concave mirror, and the second mirror in sequence; and
a data processor; wherein:
the measurement photodetector is configured to detect and process a measurement signal of the ammonia characteristic absorption line;
the reference photodetector is configured to detect and process a reference signal of the second beam passing through the reference path;
the data processor is configured to acquire a direct absorption concentration of a detected gas based on the measurement and reference signals;
a fan arranged in the case configure to accelerate a flow speed of the gas entering and exiting the ventilation hole when turned on; and
a power supply arranged in the case, electrically connected to the ammonia concentration detector and the fan, and configured to supply power to the ammonia concentration detector and the fan.

2. The semi-open atmospheric ammonia concentration detection system according to claim 1, wherein an inner surface of the case comprises a lining layer of Teflon.

3. The semi-open atmospheric ammonia concentration detection system according to claim 1, wherein:
the quantum cascade laser is configured to scan an ammonia absorption line in a standard ammonia sample;
the measurement photodetector in the measurement component is configured to capture a characteristic absorption line signal of the standard ammonia sample, and process the characteristic absorption line signal to obtain a direct absorption original spectrum;
the data processor is configured to demodulate the direct absorption original spectrum to obtain a wavelength modulation spectroscopy (WMS) modulation and demodulation absorption spectrum, obtain a baseline spectrum based on the WMS modulation and demodulation absorption spectrum, obtain spectral data based on the baseline spectrum is subtracted from the direct absorption original spectrum, and obtaining a direct absorption spectrum based on the spectral data;
the data processor is configured to obtain a direct absorption concentration of standard ammonia in a current temperature and pressure environment by using the direct absorption spectrum and an absorption spectrum with a preset concentration in a standard temperature and pressure environment; and perform, through WMS modulation and demodulation, range calibration on the direct absorption concentration of the standard ammonia in the current temperature and pressure environment, to obtain a reference spectrum that reflects an optical signal intensity; and
the data processor is configured to acquire a direct absorption concentration of atmospheric ammonia based on the measurement signal and the reference spectrum, the measurement signal being a direct absorption original spectrum corresponding to the atmospheric ammonia.

4. The semi-open atmospheric ammonia concentration detection system according to claim 3, wherein the measurement photodetector in the measurement component comprises: an measurement analog-to-digital converter and a measurement field-programmable gate array (FPGA)-based lock-in amplifier;
the measurement lock-in amplifier is configured to enhance the direct absorption spectrum and the measurement analog-to-digital converter is configured to sample the enhanced absorption spectrum to obtain the direct absorption original spectrum.

5. The semi-open atmospheric ammonia concentration detection system according to claim 4, wherein the data processor is configured to:
identify a peak position of the WMS modulation and demodulation absorption spectrum, and define a left region and a right region of the WMS modulation and demodulation absorption spectrum based on the peak position;
performing quadratic fitting on the spectral data to obtain a first fitting curve corresponding to the left region and a second fitting curve corresponding to the right region; and
connect the first fitting curve and the second fitting curve to obtain the baseline spectrum that is not affected by standard ammonia absorption.

6. The semi-open atmospheric ammonia concentration detection system according to claim 5, wherein the data processor is configured to:
obtain a K value by linearly fitting the direct absorption spectrum and the absorption spectrum with the preset concentration in the standard temperature and pressure environment;
acquire the direct absorption concentration of the standard ammonia in the standard temperature and pressure environment through the K value;
acquire a corresponding compensation coefficient M according to a temperature and an air pressure in the current temperature and pressure environment; and
obtain the direct absorption concentration of the standard ammonia in the current temperature and pressure environment through the compensation coefficient M and the direct absorption concentration of the standard ammonia in the standard temperature and pressure environment.

7. The semi-open atmospheric ammonia concentration detection system according to claim 6, wherein:
the measurement photodetector in the measurement component is configured to capture a characteristic absorption line signal of the atmospheric ammonia, and process the characteristic absorption line signal to obtain the direct absorption original spectrum;
the data processor is configured to demodulate the direct absorption original spectrum to obtain a demodulation signal corresponding to each scanning point, and normalize the demodulation signal corresponding to each scanning point to obtain a normalized spectrum, the demodulation signal comprising: a sinusoidal signal 1f and a second harmonic signal 2f; and
the data processor is configured to project the normalized spectrum to the reference spectrum to obtain a direct absorption concentration corresponding to the current normalized spectrum.

8. The semi-open atmospheric ammonia concentration detection system according to claim 7, wherein the data processor is configured to:
acquire absorbance of a maximum absorption peak in the reference spectrum;
acquire a characteristic absorption peak in the normalized spectrum, and acquire absorbance corresponding to the characteristic absorption peak;
establish a proportional relation based on the absorbance of the maximum absorption peak in the reference spectrum, a direct absorption concentration corresponding to the reference spectrum, and the absorbance corresponding to the characteristic absorption peak in the normalized spectrum, the proportional relation being
$$\frac{{S}_{A}}{{S}_{a c}} = \frac{{C}_{A}}{{C}_{a c}} ,$$
where S denotes the reference spectrum, S_{A} denotes the absorbance of the maximum absorption peak in the reference spectrum, S_{ac} denotes the direct absorption concentration corresponding to the reference spectrum, C denotes the normalized spectrum, C_{A} denotes the absorbance corresponding to the characteristic absorption peak in the normalized spectrum, and C_{ac} denotes direct absorption concentration corresponding to a to-be-solved normalized spectrum; and
obtain the direct absorption concentration corresponding to the normalized spectrum through the proportional relation.

9. The semi-open atmospheric ammonia concentration detection system according to claim 8, wherein:
the reference photodetector comprises a reference analog-to-digital converter and a reference lock-in amplifier;
the reference photodetector in the reference component is configured to detect the second beam passing through the high-concentration ammonia reaction cell to obtain the absorption spectrum corresponding to the reference component, enhance the signal of the absorption spectrum through the reference lock-in amplifier;
the reference analog-to-digital converter is configured to sample the enhanced absorption spectrum to obtain the direct absorption original spectrum;
the data processor is configured to lock a current value by using the direct absorption original spectrum and the reference component respectively, and control a drive current of the quantum cascade laser with the current value.

10. The semi-open atmospheric ammonia concentration detection system according to claim 9, wherein the data processor is configured to:
regulate an operating drive current of the quantum cascade laser, to increase from a preset minimum current until the operating drive current reaches a preset safe limit current;
acquire the direct absorption original spectrum during the regulation as a first absorption spectrum, and the direct absorption original spectrum as a second absorption spectrum, and acquire maximum absorption peaks of the first absorption spectrum and the second absorption spectrum; obtain target absorbance by using an optical signal intensity of the maximum absorption peak in the first absorption spectrum and an optical signal intensity of the maximum absorption peak in the second absorption spectrum
$$[ [ A = - {log}_{10} ( \frac{{I}_{m e a s u r e}}{{I}_{reference}} ) ] ] ,$$
and
lock a current corresponding to the target absorbance as the optimal current value, and controlling the drive current of the quantum cascade laser with the optimal current value.

11. A method, performed by a semi-open atmospheric ammonia concentration detection system, wherein:
the system comprises a case with at least one ventilation hole, an ammonia concentration detector arranged in the case and configured to detect ammonia concentration, and a fan;
the method comprises:
accelerating, by the fan, a flow speed of the gas entering and exiting the ventilation hole;
emitting, by a quantum cascade laser of the ammonia concentration detector, a laser beam and scan an atmospheric ammonia absorption line;
forming, by a reference component of the ammonia concentration detector, a reference path, the reference component comprising a high-concentration ammonia reaction cell and a reference photodetector;
forming, by a measurement component of the ammonia concentration detector, a measurement path, the measurement component comprising a first mirror, a first concave mirror, a Herriott cell, a second concave mirror, a second mirror, and a measurement photodetector;
splitting, by a beam splitter of the ammonia concentration detector, the laser beam emitted by the quantum cascade laser into a first beam passing along the measurement path and a second beam passing along the reference path, the first beam passing through the first mirror, the first concave mirror, the Herriott cell, the second concave mirror, and the second mirror in sequence; and
detecting and processing, by a data processor of the ammonia concentration detector, a measurement signal of the ammonia characteristic absorption line;
detecting and processing, by the reference photodetector, a reference signal of the second beam passing through the reference path;
acquiring, by the data processor, a direct absorption concentration of a detected gas based on the measurement and reference signals.
